(19) United States
(12) Patent Application Publication
(10) Pub. No.: US 2013/0090639 A1
(43) Pub. Date: Apr. 11, 2013

(54) SYSTEM AND METHODS FOR DESTRUCTION OF TISSUE USING CRYOGENIC FLUID

(71) Applicants: Joseph Atias, Great Neck, NY (US); Martin C. von Dyck, Upper Black Eddy, PA (US)

(72) Inventors: Joseph Atias, Great Neck, NY (US); Martin C. von Dyck, Upper Black Eddy, PA (US)

(21) Appl. No.: 13/625,855

(22) Filed: Sep. 24, 2012

Related U.S. Application Data
(60) Provisional application No. 61/538,845, filed on Sep. 24, 2011.

(51) Int. Cl. A61B 18/02

(52) U.S. Cl. CPC A61B 18/02
USPC 606/23

(57) ABSTRACT

Provided are systems and methods for destroying tissue using a cryogenic fluid. The systems generally comprise a hollow outer cylinder; a hollow inner cylinder placed within the hollow outer cylinder; a tip at the front end or distal end of the outer cylinder; wherein a cryogenic fluid is made to flow through the hollow region of the inner cylinder which oozes out or flows out of more than one grooves or apertures on the surface of the inner cylinder and then passes through the hollow portion of the outer cylinder. Yet another preferred embodiment provides for a housing component having a combination of storage for additional system cooling and exhaust facilities.

### CROSS-REFERENCE TO RELATED APPLICATIONS

This claims benefit of U.S. Provisional Patent Application No. 61/538,845 filed Sep. 24, 2011, the entirety of which is incorporated herein by reference.

### FIELD OF THE INVENTION

The present invention relates to the field of cryogenic fluids and cryoprobes, and related systems and methods.

### BACKGROUND

Cryogenic fluids have been used for various purposes including medical treatment of cancer, scars, keloids, unwanted skin growth, glaucoma and eye diseases, using their property to freeze and destroy tissue due to the temperature effect. One of the modes of using the cryogenic fluids for effecting the said treatments is by using systems or devices which are inserted or incised into the region containing unwanted tissue or tissues, and cryogenic fluids are made to flow within the inner region of the system or device and thereby passing the cooling or freezing effect through the system or device to the unwanted tissue surrounding the system or device, thereby destroying the tissue using a freezing effect. Such systems or device are also commonly known as Cryoprobes.

Cryoprobes that have been developed till date and which are in practice have certain drawbacks and scope for improvement. One such drawback is the lack of a system which provides a multiple outlet mechanism for a cryogenic fluid within the system which will improve fluid flow and circulation, avoid blockages, cause faster cooling, better distribution of cooling effect, increase efficiency, increase longevity and durability of the system, and consequently save cost of treatment which shall benefit both medical practitioners as well as the patients or subjects.

Another drawback and area of improvement in cryoprobes relates to the exhaust mechanism of the gaseous form of the cryogenic liquid after its use for the purpose of cooling. There are a few exhaust mechanisms available but none of those provide a combination of multiple outlet pores for the effective and quick escaping of gases, and also provide a way to use the temperature or coolness of the escaping gas to increase efficiency of the system.

In yet another drawback and area of improvement in cryoprobes, the cryogenic fluid storage and supply mechanism is to be considered. Most cryoprobes store cryogenic fluids externally and supply the fluid inside the system through a tube at the time of the treatment. There is no existing mechanism that allows the storage of the fluid in a container which is connected to the system, and through which the fluid is supplied inside the system at the time of treatment. This container may also optionally act as a handle and balance out the heat received by the hands or any device used to hold the system by a medical practitioner, thus increasing the overall heat efficiency of the system translating in lower time and lower cost of treatment.

The present invention in its various embodiments addresses the above and other possible drawbacks and limitations of the currently used systems and methods for destroying tissue using cryogenic fluids.

### SUMMARY

It is an object of this disclosure to provide cryoprobe systems with a more efficient cryogenic fluid pathway via multiple outlet mechanism for supply of a cryogenic fluid within the system.

It is yet another object of the present disclosure to provide cryoprobe systems with a component carrying out the dual function of housing an efficient exhaust mechanism and also optionally acting as a storage container for the cryogenic fluids, the component being connected to one or more of other components of the system.

According to a first aspect of this disclosure, there are provided systems for destroying tissue using a cryogenic fluid, the systems generally comprising a hollow outer cylinder; a hollow inner cylinder placed within the hollow outer cylinder; a tip at the front end or distal end of the outer cylinder; wherein a cryogenic fluid is made to flow through the hollow region of the inner cylinder which flows out of more than one grooves or apertures on the surface of the inner cylinder and then passes through the hollow portion of the outer cylinder, thereby cooling the walls of the outer cylinder from where the cooling effect is passed on to the tissue to be destroyed.

In another aspect, provided are systems for destroying tissue using a cryogenic fluid, the systems generally comprising a housing component having a combination of storage and exhaust facilities. The housing component may be placed or connected towards the proximal end or tail end of the cryoprobe system, and comprises o a storage portion which is used to store the cryogenic fluid or exhaust fluid that shall be used for passing through the cryoprobe system at the time of treatment; and an exhaust system which has multiple outlets for allowing the exhaust gases to escape in an effective and properly distributed manner, thereby increasing outflow rate and avoiding blockages. The housing system may also be used as a handle and can thus replace a conventional handle of a cryoprobe system.

According to another aspect of this disclosure, also provided are cryoprobe systems for destroying tissue using a cryogenic fluid, said systems generally comprise a hollow outer cylinder and a hollow inner cylinder placed within said hollow outer cylinder, said hollow outer cylinder comprising a tip at its operative distal end, said hollow inner cylinder and said hollow outer cylinder being open at their operative proximal ends, characterized in that, said hollow inner cylinder comprising more than one grooves or apertures located near its operative distal region, said grooves or apertures being circumferential grooves or apertures in a spaced apart manner with respect to each other.

In one embodiment, said hollow inner cylinder is substantially co-axial to said hollow outer cylinder.

In one embodiment, said hollow inner cylinder is substantially co-axial to said hollow outer cylinder.

In one embodiment, said grooves are non-collinear grooves.

In one embodiment, said tip is sharp and pointed, adapted to act as a needle for incision or insertion of said system into a desired area or portion of the body of a subject.

In one embodiment, tip comprises a solid interior part adapted to receive the operative distal end of said inner cylinder so that it forms a fixed substantially co-axial assembly with said outer cylinder.

In one embodiment, said system comprises a housing component having a combination of storage and exhaust facilities, said housing component being placed or connected towards the operative proximal end of said cryoprobe system, and further comprising a storage portion which is used to store the cryogenic fluid to be used for passing through said cryoprobe system at the time of treatment; and an exhaust system having multiple outlets or vents for allowing exhaust gases to escape in an effective and properly distributed manner, thereby increasing outflow rate and avoiding blockages.

In one embodiment, said system comprises a housing component adapted to grip said system, said housing being a dual body assembly.

In one embodiment, said system comprises a housing component adapted to grip said system, said housing being a dual body assembly with ribbed and/or dimpled surfaces for gripping.

In one embodiment, said system comprises a housing component having a combination of storage for additional system cooling and exhaust facilities, said housing component comprising a plurality of laterally located holes for exhaust of said fluid.

In one embodiment, said system comprises a housing component having a combination of storage and exhaust facilities, said housing component adapted to receive open end portion of said hollow inner cylinder and open end portion of said hollow outer cylinder.

Preferably, angle of said tip is less than 14 degrees.

Preferably, the outer surface of said system is coated with one or more substances such as a hydrophilic substance or any other lubricant substance for ease of cryoprobe insertion and cryoprobe removal.

In another aspect of this disclosure, there are also provided methods for destroying tissue using a cryoprobe system using a cryogenic fluid.

In one embodiment the method comprises the steps of:

a) inserting a portion of said system, with its tip, into a lesion or desired area or portion of the body of a subject, such that the tissue to be destroyed are surrounding the walls of the outer cylinder of the system; and

b) allowing cryogenic fluid to flow, in a first direction, towards the tip through a hollow region of an inner cylinder of said system, said inner cylinder placed within said hollow outer cylinder, said hollow outer cylinder comprising said tip at its operative distal end, said hollow inner cylinder and said hollow outer cylinder being open at their operative proximal ends, characterized in that, said hollow inner cylinder comprising more than one grooves or apertures located near its operative distal region, said grooves or apertures being circumferential grooves or apertures in a spaced apart manner with respect to each other such that said fluid flows out of said grooves or apertures and enters the passage formed by the outer wall of said inner cylinder and the inner wall of said outer cylinder, thereby cooling the walls of the outer cylinder from where the cooling effect is passed on to the tissue to be destroyed, to flow in a second direction towards an exhaust port in a housing component placed in an operative proximal end of said system.

### BRIEF DESCRIPTION OF THE DRAWINGS

FIG. 1 is a cross-sectional view of the distal end or the front end of an illustrative example of an embodiment of a cryoprobe system.

FIG. 2 is an external view of the housing component of the base system (placed or connected towards the proximal or tail end) of an illustrative example of an embodiment of a cryoprobe system.

FIG. 3 is an illustrative example of the tip of a cryoprobe system. Shown is an internal view of the inner cylinder lodged in the solid portion of the tip of the outer cylinder; the cryofluid flow through the grooves or apertures of the inner cylinder is illustrated.

FIG. 4 is an illustrative example of an embodiment of a cryoprobe system showing an external isometric view of the system.

FIG. 5 is an illustrative example of an embodiment of a cryoprobe system showing an internal view of the housing component placed or connected towards the proximal or tail end of the system.

FIG. 6a is an illustrative example of the vented housing component shown in isometric view.

FIG. 6b illustrates a top cut-section view of the vented housing component of a cryoprobe system. The directional arrows depict the flow of the cryogenic vapors or gas from the housing component.

FIG. 7 is an illustrative example of a first part of the dual assembly of the housing component of FIG. 6 of the cryoprobe system in top view.

FIG. 8 is an illustrative example of a second part of the dual assembly of the housing component of FIG. 6 of the cryoprobe system in isometric view.

### DETAILED DESCRIPTION OF THE DIAGRAMS

The present invention, in a preferred embodiment, provides a system for destroying tissue using a cryogenic fluid.

In various embodiments, such systems are referred to herein as cryoprobe systems. The term ‘cryoprobe system’ refers to systems that use cryogenic fluids to destroy tissue, which may or may not have the features of one or more of the systems illustrated in one or more of the embodiments of the present disclosure.

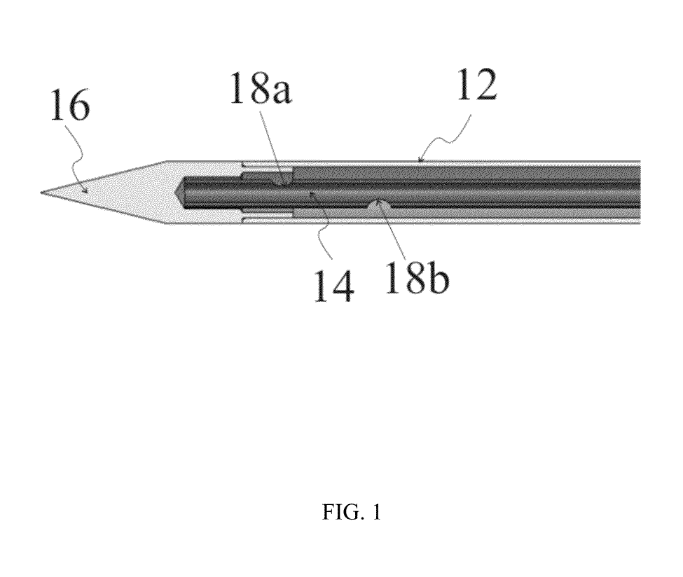

FIG. 1 is an illustrative example of an embodiment of a system (100) in accordance to the present invention providing a cross-sectional view of the operative distal end or the operative front end of the system. Reference numeral 16 represents a cutting and/or piercing tip with a receptacle for the inner hollow cannula (14), allowing for the centering of the inner hollow cannula (14) within the outer hollow cannula (12). Reference numerals 18a and 18b show the grooves or apertures for the high capacity fluid flow into the outer hollow cannula (12).

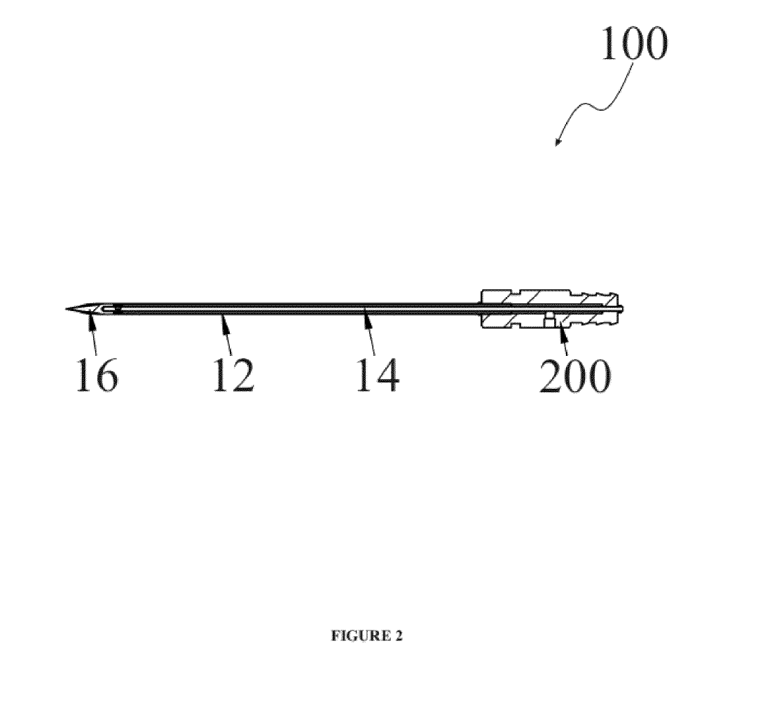

FIG. 2 is an illustrative example of an embodiment of a system (100) in accordance to the present invention providing an external view of the entire system the vented housing component (200) placed or connected towards the proximal or tail end of the system.

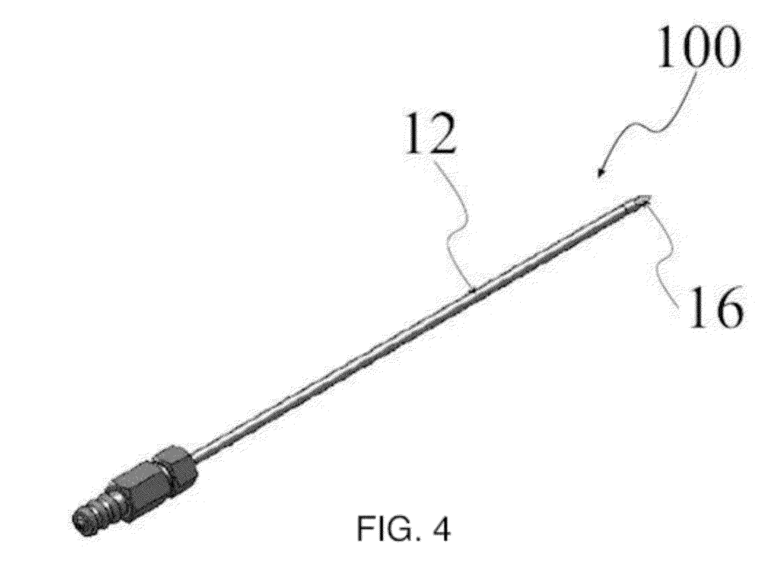

FIG. 4 is an illustrative example of an embodiment of a system (100) in accordance to the present invention providing an external isometric view of the system.

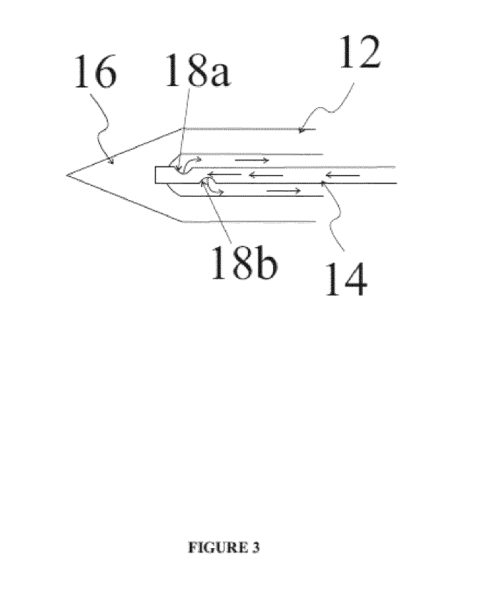

FIG. 3 is an illustrative example of the tip of the system (100) in accordance to the present invention provided an internal view of the inner cylinder lodged in the solid portion of the top of the outer cylinder, and illustrates the cryofluid flow through the grooves or apertures of the inner cylinder.

In one embodiment, the system comprises a hollow outer cylinder (12) and a hollow inner cylinder (14) placed within the hollow outer cylinder (12). The hollow outer cylinder (12) comprises a tip (16) at its operative front end or operative distal end. The hollow inner cylinder (14) is open at its operative proximal end. The hollow outer cylinder (12) is open at its operative proximal end. The hollow inner cylinder (14) is substantially, without limitation, co-axial to the hollow outer cylinder (12). A cryogenic fluid is made to flow, in a first direction, towards the tip (16), through the hollow region of the inner cylinder (14).

In another embodiment the system comprises more than one grooves (18a, 18b) or apertures or ports, located near the operative distal region, on the inner cylinder (14). The grooves (18a, 18b) or apertures are, preferably, non-collinear and circumferentially placed about the inner hollow cylinder (14) in a spaced apart manner with respect to each other. The cryogenic fluid flows out of these more than one grooves (18a, 18b) or apertures or ports and then passes, in a second direction, opposite to the first direction, through the hollow portion of the outer cylinder (12) made by the gap between the outer wall of the inner cylinder (14) and the inner wall of the outer cylinder (12), thereby cooling the walls of the outer cylinder (12) from where the cooling effect is passed on to the tissue to be destroyed. The tip (16) at the operative front end is preferably sharp and pointed, and acts as a needle for incision or insertion of the system (100) into a desired area or portion of the body of a subject. At its interior part, the tip (16) is solid and receives the operative distal end of the inner cylinder (14) so that it forms a fixed substantially co-axial assembly with the outer cylinder (12).

In another aspect, methods of using the cryoprobe systems in accordance with this disclosure are provided. In one embodiment, the system (100) is inserted into a lesion or desired area or portion of the body of a subject, such that the tissue to be destroyed are surrounding the walls of the outer cylinder of the system, and when the cryogenic liquid passes through the hollow portion of the outer cylinder, the freezing effect is passed on to the tissue through the walls of the outer cylinder, which cause the tissue to be destroyed due to the extremely low (cryogenic) temperature. The system and methods, in the aforementioned preferred embodiments, specifically uses two or more grooves or apertures on the surface of the inner tube instead of a single opening at the distal end of the inner tube, and due to the two or more apertures or grooves, the flow of the cryogenic fluid into the hollow portion of the outer cylinder is well distributed and more uniform leading to enhanced flow efficiency, enhanced heat efficiency, reduction or elimination of blockages, faster cooling and reduced cost of treatment.

In another embodiment, the tip (16) may be an extension of the outer cylinder (12) and can have a hollow portion, which when filled by a cryogenic fluid will have the same effect and function as the outer cylinder (12) for the purpose of this invention. In a preferred embodiment the angle of the tip (16) is below 14 degrees.

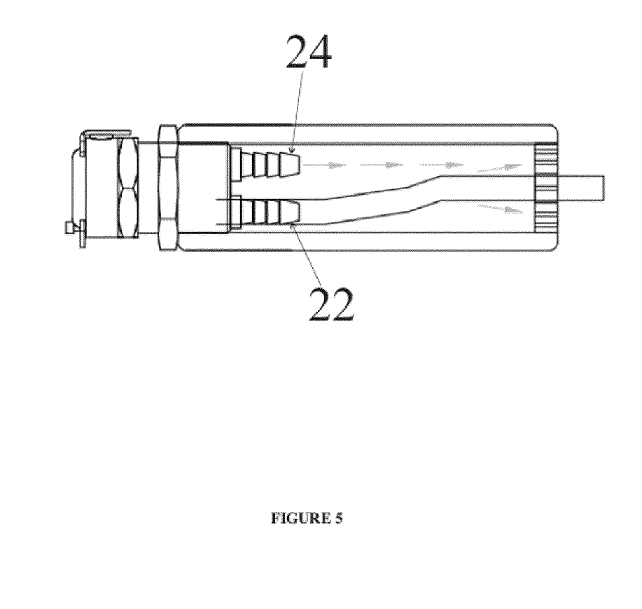

FIG. 5 is an illustrative example of an embodiment of a cryoprobe system in accordance with the present disclosure. Reference numeral 22 refers to an inlet port for supplying cryogenic fluid to the inner cylinder (14). Reference numeral 24 refers to an outlet port for receiving fluid from the outer cylinder (12). The arrows depict the second direction of the fluid through the outlet port.

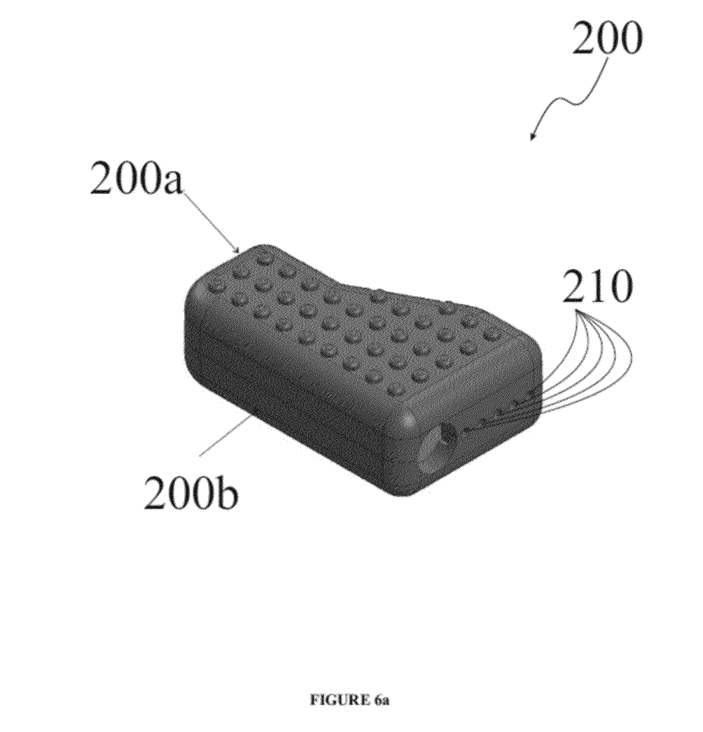

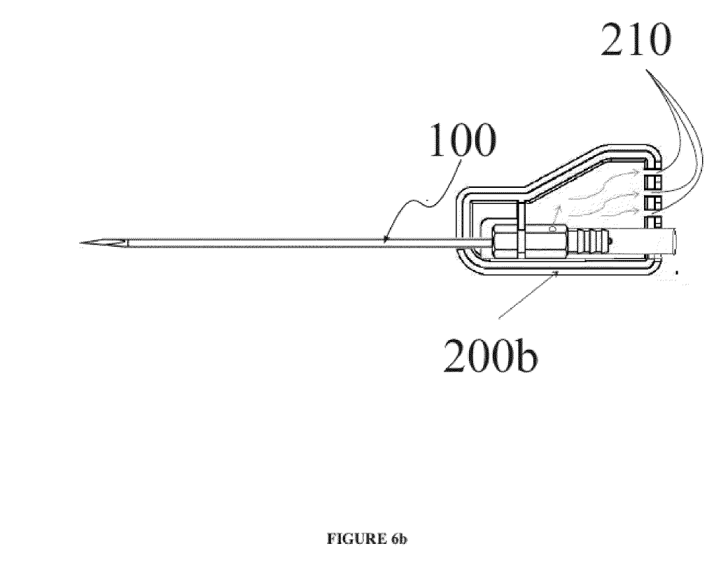

FIG. 6a is an illustrative example of the vented housing component (200) of the system shown in isometric view. The vents are displayed by reference numerals 210 at the operative proximal end of the housing component. These vents provide an exit airway passage for exiting the cryogenic vapors or gas.

FIG. 6b illustrates a top cut-section view of the vented housing component (200) of the system. The directional arrows depict the flow of the cryogenic vapors or gas from the component.

In an additional aspect provided is a cryoprobe system (100) that comprises a housing component (200) having a combination of storage and exhaust facilities. The housing component (200) may be placed or connected towards the operative proximal end or operative tail end of the cryoprobe system (100), and comprises a storage portion which is used to store the cryogenic fluid that shall be used for passing through the cryoprobe system at the time of treatment; and an exhaust system which has multiple outlets for allowing the exhaust gases to escape in an effective and properly distributed manner, thereby increasing outflow rate and avoiding blockages. The exhaust system may be placed in the housing component in such a manner that by using a heat exchanging mechanism, the coolness of the outgoing gas can be made use of, and in a way recycled back into the system, thereby making maximum use of heat exchange leading to enhanced system efficiency. The housing system may also be used as a handle and can thus replace a conventional handle of a cryoprobe system, which when held using a hand or any other device by a medical practitioner or user of a cryoprobe system, will assist in reducing the effect of heat transferred to the system by the externally placed hand or device, which shall thus lead to increased system efficiency in comparison to a cryoprobe system with a conventional handle and an external source of cryogenic fluid. The housing, preferably, is a dual body assembly with ribbed surfaces for gripping.

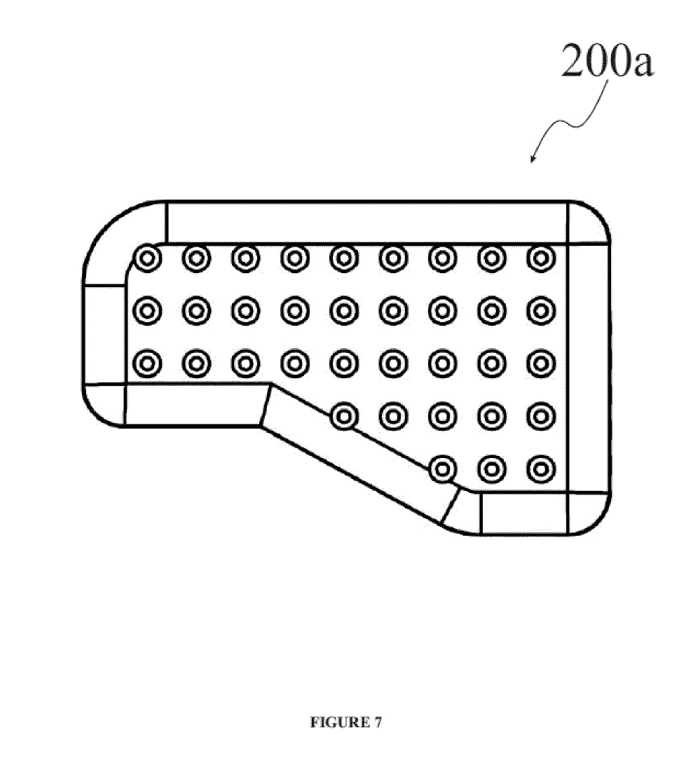

FIG. 7 is an illustrative example of a first part (200a) of the dual assembly of the housing component (200) of FIG. 6 of the system shown in a top view.

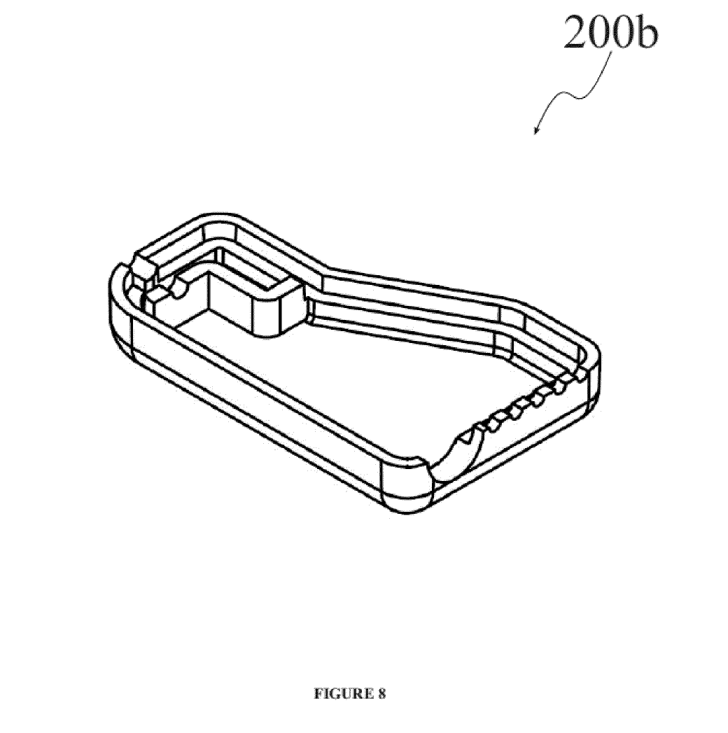

FIG. 8 is an illustrative example of a second part (200b) of the dual assembly of the housing component (200) of FIG. 6 of the system in isometric view.

The systems and methods disclosed herein may be used for treatment of a subject including human beings or animals other than human beings. In one embodiment the systems and methods may be used for clinical studies or clinical testing or clinical research.

In one embodiment the systems and methods may be used for treatment of cancer or cancer cells, for example skin cancer (such as basal cell carcinoma and others), renal cell carcinoma, breast cancer, liver cancer, prostate cancer, lung cancer, pancreas cancer, bladder cancer, colon cancer, stomach cancer or a combination thereof.

In another embodiment the systems and methods may be used for treatment of skin conditions including diseases, disorders, conditions or ailments affecting any one or more layers of dermis or sub-dermis. In another embodiment the systems and methods disclosed herein may be used for cosmetic purposes or cosmetic enhancements of physical appearance of a subject, including for removal of any unwanted skin marking, coloration, or the like whether naturally occurring or man-made.

In other embodiments the systems and methods disclosed herein may be used for treatment of eye related diseases or disorders.

In certain embodiments, the systems and methods disclosed herein may be associated with or used in connection with one or more computer programs which may have a predefined algorithm or logic or program or code associated with the functioning of the said systems.

The term “cryogenic fluid” for the purpose herein means a liquid or a gas which can flow and in the state of being a gas or liquid has temperature lower than −80° C., and includes but is not limited to forms of argon, oxygen, ammonia, nitrogen, neon, krypton, dichlorodifluoromethane, chlorodifluoromethane, chloropentafluoroethane, xenon, trichlorofluoromethane, tetrafluoromethane, sulfur hexafluoride, perfluoropentane, or any combination thereof.

In one embodiment, any solid, liquid, gaseous or other state of matter of any substance, material or particle may be added to cryogenic fluid for improving and/or influencing its properties such as but not limited to temperature, density, viscosity, specific gravity, pH, friction, mechanical stability, corrosiveness, color, longevity, latent heat of vaporization, latent heat of condensation, latent heat of freezing, or any combination thereof.

In one embodiment, any materials may be used for construction or fabrication of one or more components of the systems disclosed herein provided it is suitable for performing the function as required for and by one or more component of the system, and may include but is not limited to a metal, a metal alloy, a polymer, rubber, glass, minerals, gem stones, fiber, ceramic, or any combination thereof.

In one embodiment, the one or more parts or components of the system may be connected and fixed, or may be detachable and re-attachable. Detachable components can be attached or fixed with one or more of other components using mechanisms such as but not limited to screw threads, twist and lock mechanism, magnetic locking, vacuum induced locking, friction fit, snap fit, or any combination thereof.

In one embodiment the tip of the cryoprobe system may be used for application or insertion of some additional medicine during a treatment using the systems and methods disclosed herein.

In another embodiment, the systems and methods may be employed in the form of or in conjunction with nano-particles or nano-devices while performing nano-cryosurgery.

In one embodiment the systems may be inserted into or completely through a lesion or a desired area or portion of the body of a subject through any angle ranging from 0 to 360 in terms of any of three-dimensional axis such as length, breadth and height.

In one embodiment the hollow outer or hollow inner cylinder need not be perfectly cylindrical in shape. Surfaces of the inner or outer cylinder may have irregular surfaces or tapering surface, or may be semi-conical or any other shape provided that the function of a cylindrical tube can be performed by the said components.

In one embodiment the hollow outer or hollow inner cylinder may be either rigid or flexible.

In another embodiment, the systems may include applied coloring, e.g. with colorants such as dyes or pigments or paint for providing an aesthetic view to the system.

In an embodiment the systems and methods disclosed herein may be combined with one or more of additional therapies of cancer treatment such as but not limited to applying electric signals, applying magnetic field, applying radioactive waves, applying chemicals, applying thermal energy, applying a vibrational effect. One or more of these therapies may be used either independently or together with the disclosed systems or methods by making suitable modification(s) to the systems. The vibrational effect may be provided using vibrational mechanisms such as but not limited to those mentioned in patent application US 2010/0198206 to Alexander Levin.

In one embodiment, the systems may have included an embedded or externally attached clock or stopwatch on the external body of the system to indicate various time related data such as but not limited to time elapsed, time remaining, current time in one or more time zones, or a combination thereof. Such clocks may be digital or analog.

In an embodiment the systems and methods may allow the exhaust gas to escape in air or to be collected in a separate container air.

In an embodiment the systems and methods may provide for the housing component to have more than one compartments used to store one or more types of cryogenic fluids.

In an embodiment the systems and methods may install, embed or connect one or more sensors to the system, such as but not limited to, temperature sensors, chemical sensors, gas leak sensors, internal damage sensors, or a combination thereof. The sensors may be connected to a computing device or programmable logic controller, proportional—integral—derivative controllers. The combination of sensors and controllers may be used to monitor and control the methods disclosed.

In one embodiment more than one system or methods disclosed can be used at the same time.

In another embodiment a combination system can be composed of multiple cryoprobe systems, at least one of which shall be a system in accordance with the present invention, and the combination system can be used to destroy tissue using cryogenic fluids.

In one embodiment the systems and methods may be enabled through a computer program that may be embodied in a computer.

In another embodiment the systems and methods may enable a mechanism to control or modify the temperature of the system or the temperature of the cryogenic fluid by:

a) enabling a warming feature in the system, in patent U.S. Pat. No. 5,800,488 to Crockettor

b) controlling the flow of the cryogenic fluid, or

c) adding of any material to the system or the cryogenic fluid, or a combination thereof.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude or rule out the presence or addition of one or more other features, integers, steps, operations, elements, components, and/or groups thereof.

The process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously, in parallel, or concurrently.

In addition to the embodiments and examples shown, numerous variations are possible, which may be evident to a person skilled in the arts relating to this disclosure.

1. A cryoprobe system for destroying tissue using a cryogenic fluid, said system comprising:
a hollow outer cylinder and a hollow inner cylinder placed within said hollow outer cylinder, said hollow outer cylinder comprising a tip at its operative distal end, said hollow inner cylinder and said hollow outer cylinder being open at their operative proximal ends, characterised in that, said hollow inner cylinder comprising more than one grooves or apertures located near its operative distal region, said grooves or apertures being circumferential grooves or apertures in a spaced apart manner with respect to each other.

2. A system as claimed in claim 1 wherein, said grooves or apertures are non-collinear grooves or apertures.

3. A system as claimed in claim 1 wherein, said tip is sharp and pointed, adapted to act as a needle for incision or insertion of said system into a desired area or portion of the body of a subject.

4. A system as claimed in claim 1 wherein, tip comprising a solid interior part adapted to receive the operative distal end of said inner cylinder so that it forms a fixed substantially co-axial assembly with said outer cylinder.

5. A system as claimed in claim 1 wherein, said system comprising a housing component having a combination of storage and exhaust facilities, said housing component being placed or connected towards the operative proximal end of said cryoprobe system, and further comprising a storage portion which is used to store the cryogenic fluid to be used for passing through said cryoprobe system at the time of treatment; and an exhaust system having multiple outlets or vents for allowing exhaust gases to escape in an effective and properly distributed manner, thereby increasing outflow rate and avoiding blockages.

6. A system as claimed in claim 1 wherein, said system comprising a housing component adapted to grip said system, said housing being a dual body assembly.

7. A system as claimed in claim 1 wherein, said system comprising a housing component adapted to grip said system, said housing being a dual body assembly with ribbed and/or dimpled surfaces for gripping.

8. A system as claimed in claim 1 wherein, said system comprising a housing component having a combination of storage for additional system cooling and exhaust facilities, said housing component comprising a plurality of laterally located holes for exhaust of said fluid.

9. A system as claimed in claim 1 wherein, said system comprising a housing component having a combination of storage and exhaust facilities, said housing component adapted to receive open end portion of said hollow inner cylinder and open end portion of said hollow outer cylinder.

10. A system as claimed in claim 1 wherein, angle of said tip is below 14 degrees.

11. A system as claimed in claim 1 wherein, the outer surface of said system is coated with a substance selected from a group of substances consisting of a hydrophilic substance or any other lubricant substance for ease of cryoprobe insertion and cryoprobe removal.

12. A method for destroying tissue using a cryoprobe system using a cryogenic fluid, said method comprising the steps of:
a) inserting a portion of said system, with its tip, into a lesion or desired area or portion of the body of a subject, such that the tissue to be destroyed are surrounding the walls of the outer cylinder of the system;
b) allowing cryogenic fluid to flow, in a first direction, towards the tip through a hollow region of an inner cylinder of said system, said inner cylinder placed within said hollow outer cylinder, said hollow outer cylinder comprising said tip at its operative distal end, said hollow inner cylinder and said hollow outer cylinder being open at their operative proximal ends, characterised in that, said hollow inner cylinder comprising more than one grooves or apertures located near its operative distal region, said grooves or apertures being circumferential grooves or apertures in a spaced apart manner with respect to each other such that said fluid flows out of said grooves or apertures and enters the passage formed by the outer wall of said inner cylinder and the inner wall of said outer cylinder, thereby cooling the walls of the outer cylinder from where the cooling effect is passed on to the tissue to be destroyed, to flow in a second direction towards an exhaust port in a housing component placed in an operative proximal end of said system.
